(19) United States
(12) Patent Application Publication
(10) Pub. No.: US 2003/0130346 A1
(43) Pub. Date: Jul. 10, 2003

(54) Use of medium-chain triglycerides for the prevention and therapy of adiposity

(75) Inventors: Lubomir Kuzela, Praha (CZ); Walter Feldheim, Kronshagen (DE)

Correspondence Address: BIRCH STEWART KOLASCH & BIRCH, PO BOX 747, FALLS CHURCH, VA, 22040-0747, US

(73) Assignee: Horst Heirler, Worthsee (DE)

(21) Appl. No.: 10/180,834

(22) Filed: Jun. 25, 2002

(30) Foreign Application Priority Data
Jun. 25, 2001 (DE) DE 101 30 491.9

(51) Int. Cl. A61K 31/225; A61K 31/202

(52) U.S. Cl. 514/547; 514/560

(57) ABSTRACT

Use of medium-chain triglycerides for the prevention and therapy of adiposity The use of medium-chain triglycerides or of a composition containing said medium-chain triglycerides for the prevention or therapy of overweight or adiposity is described. Said composition preferably also contains long-chain essential triglycerides, preferably α-linoleic acid and/or linolenic acid as well as, optionally, further components and/or additives.

This nonprovisional application claims priority under 35 U.S.C. §119(a) on Patent Application No. DE 101 30 491.9 filed in Germany on Jun. 25, 2001, which is herein incorporated by reference.

The present invention relates to the use of medium-chain triglycerides (MCT) or a composition containing medium-chain triglycerides, for example a dietetic foodstuff for the prevention or therapy of overweight or adiposity. This composition preferably also contains long-chain essential triglycerides, preferably a-linoleic acid and/or α-linolenic acid, as well as optionally further components and/or additives.

In the affluent industrial nations, the biggest problem concerning nutrition is overnutrition. The constantly rising number of persons suffering from overweight or adiposity, a considerable share of which are children or adolescents, is problematic due to its consequence, namely the increase in nutrition-related diseases. Overweight is a risk factor for diseases of the skeletal and musculoskeletal system, hypertension (4-fold risk), type 2 diabetes mellitus (6-fold risk), heart attack (4-fold risk), breast cancer (3-fold risk), biliary stones (10-fold risk), gout etc.

Overnutition is due to an excessive uptake of energy from food, exceeding the daily energy need. Due to a higher share in fats and a reduced uptake of carbohydrates, the change in nutritional habits leads to a higher energy content in food. Moreover, the energy need has been sinking continuously as the energy used for physical activity in connection with a person's professional occupation or spare time activity has decreased.

Until now, there has not been a useful concept, which could change the everyday situation fundamentally. Too sharp a reduction of food uptake over a longer period of time is not accepted as food, which means quality of life, is always accessible and can be obtained cheaply. Until now, a change in nutrition habits by returning to a higher share of vegetable foodstuffs in one's diet is also refused.

Furthermore, the known therapies for people suffering from overweight or adiposity are not satisfactory and have a number of side effects. Thus, the known therapeutical forms are various special diets, partly having extreme ratios of nutrients, and drugs having side effects. Special diets require relevant knowledge and, if they deviate from the standard to a higher degree, a higher stamina, and in many cases, above all, if they are followed for a longer period of time, they do not secure a sufficient uptake of nutrients. Especially if the uptake of fat is reduced sharply or even to an extreme extent, the provision with the fat-soluble vitamins and important nutrients like prostaglandins and immunoglobulins from essential fatty acids is not secured and the role which fat plays within the metabolism, e.g. with respect to the resorption of calcium and the synthesis and release of serotonine, is disregarded. Known side effects of medicaments are steatorrhea, flatulence, diarrhoea and an increase in blood pressure.

However, due to the costs and the side effects involved in a therapy with drugs, prevention/therapy by correspondingly co-ordinated foodstuffs would be preferable.

Thus, the technical problem underlying the present invention is to provide means for the treatment/prevention of overweight or adiposity, which do not have the disadvantages discussed above.

The technical problem is solved by providing the embodiments characterised in the claims.

In a broadly-based study described in the following examples, in which test persons, in the course of the day, were provided with two controlled dietary samples ad libitum in three meals, the dietary samples differing with respect to the content in medium-chain triglycerides (MTC), it could be demonstrated that these fatty acids are suitable for the prevention or therapy of overweight and adiposity. To sum up, the following conclusions can be drawn from this study:

1. With respect to persons with normal weight, the uptake of energy from food can be increased when consuming MCT-containing foodstuffs (compared to long-chain fatty acids (LCT)-containing products) without increasing the physical weight. Thus, in the case of persons with normal weight, an increase in the share of MCT-containing foodstuffs in the diet can be used as a prophylactic measure against overweight.

2. In the case of overweight persons wanting to reduce their weight with the help of a reduction diet (e.g. 1200 kcal/day), the stamina can be increased with MCT-containing foodstuffs—also by more fat as taste carrier—because physical weight is further reduced despite a higher uptake of energy and thus this seems to be a sensible therapeutic measure for overweight persons.

3. The result is the surprising possibility of using MCT-foodstuffs for persons with normal weight and for overweight persons as part of a normal diet or a reduction diet.

Thus, the present invention relates to the use of medium-chain triglycerides or a composition containing medium-chain triglycerides, e.g. as dietary foodstuff, for the prevention or therapy of overweight or adiposity.

The term “medium-chain triglycerides” as used herein refers to triglycerides, essentially containing almost only caprylic acid (C8:0) and/or capric acid (C10:0) according to the usual chemical nomenclature.

The person skilled in the art knows sources for medium-chain triglycerides, preferably coconut oil or palm oil.

The exclusive use of MCT-oils as part of the diet is limited. Thus, the administration of MCTs in the form of MCT-containing foodstuffs, e.g. margarine, is recommendable so that the consumer has a lot of possibilities of using MCT-containing foodstuffs instead of LCT-containing foodstuffs.

For the therapy/prevention of overweight or adiposity, the medium-chain triglycerides or the composition containing these are/is preferably offered in the form of foodstuffs known to the consumer, optionally containing further additives, e.g. essential triglycerides or corresponding compositions, instead of diets without medium-chain triglycerides or pharmaceutical compositions.

When sharply reducing LCTs in a diet, it has to be taken into account that essential triglycerides are usually also administered with these fats. Consequently, MCT-products should contain these vital fafty acids in a sufficient amount. The resorption of fat-soluble vitamins also depends on the amount of LCTs in the diet. For this reason, too, suitable products with MCT should contain a minimum amount of LCT.

Thus, in a preferred embodiment of the use according to the invention, the composition contains in the fat phase (a) 70 to 90% medium-chain triglycerides, (b) α-linoleic acid and/or (c) α-linolenic acid.

Even more preferred is an embodiment in which the fat phase of the composition additionally contains γ-linolenic acid, a content of between 1 and 2.5% being most preferred.

In a further preferred embodiment, the content of α-linoleic acid is 3.8 to 13.4%.

In a further preferred embodiment, the composition contains 3 to 8% α-linolenic acid.

In yet another preferred embodiment, the content of saturated long-chain triglycerides is not higher than 2.5%.

The medium-chain triglycerides contained in an amount of 70 to 90% in the fat phase of the composition for the use according to the invention are preferably caprylic acid and capric acid.

The saturated long-chain triglycerides contained in an amount of 0.9 to 2.5% in the composition for the use according to the invention are preferably derived from safflor oil in an amount of 0.5 to 1.5%, from linseed oil in an amount of 0.3 to 0.7% and from an emulgator, e.g. Lecidan in an amount of 0.1 to 0.3%.

The α-linoleic acid, contained in the composition in an amount of 3.8 to 13.4% is preferably derived from safflor oil in an amount of 3 to 11% and from linseed oil in an amount of 0.8 to 2.4%. Linseed oil is also a preferred source of α-linolenic acid which is contained in an amount of 3 to 8%. Borage oil is a preferred source of γ-linolenic acid which is contained in the composition in an amount of 1 to 2.5%.

The fatty acids contained in the composition in an amount of 1.3 to 3.6% for the use according to the invention are preferably derived from safflor oil in an amount of 0.5 to 1.5% and from linseed oil in an amount of 0.8 to 2.1%.

In a particularly preferred embodiment, the composition of the fat phase has the following composition:

| | |
|---|---|
| medium-chain triglycerides | 70-90% |
| saturated, long-chain triglycerides | 0.9-2.5% |
| α-linoleic acid | 3.8-13.4% |
| α-linolenic acid | 3-8% |
| γ-linolenic acid | 1-2.5% |
| oleic acid | 1.3-3.6%. |

In an even more preferred embodiment, the composition of the fat phase has the following composition:

| | |
|---|---|
| medium-chain triglycerides | 80% |
| saturated, long-chain triglycerides | approx. 1.6% |
| α-linoleic acid | approx. 9% |
| α-linolenic acid | approx. 5.4% |
| γ-linolenic acid | approx. 1.8% |
| oleic acid | approx. 2.2%. |

In a further preferred embodiment, the composition for the use according to the invention moreover contains, in addition to the triglycerides indicated above, emulsifiers, fat-soluble vitamins, β-carotene and/or lecithin. It is, for example, possible to use Lecidan SB (emulsifier consisting of mono- and diglycerides as well as lecithin) as emulsifier in a concentration of 0.5%. In a particularly preferred embodiment the fat phase of the composition contains the vitamins A, D and/or E.

Preferably, the fat phase of the composition for the use according to the invention accounts for 80% and the aqueous phase for 20%, wherein, in a preferred embodiment, the aqueous phase contains vitamin C, folic acid and/or vitamin B12.

For margarine, the preferred amounts of vitamins per 100 g fat are 0 to 2 mg vitamin A, 0 to 300 ng β-carotene, 0 to 50 μg vitamin D, 0 to 100 mg total tocopherol, 0 to 1 μg vitamin B12, 0 to 5 mg folic acid and 0 to 75 mg vitamin C. The resorption of calcium and iron is improved by adding vitamin C.

### EXAMPLE 1

### Administration of Medium-Chain Triglycerides in the Form of Margarine

Margarine is the preferred form in which medium-chain triglycerides are appropriately placed on the market together with further ingredients. It contains approx. 18% w/v water, at least 80% w/v fat and approx. 2% w/v dry substance. The ratio of aqueous phase to fat phase is approx. 20%:80%.

Production

1. The water-soluble components (vitamin B12, vitamin C, folic acid, aroma) are diluted in water and mixed. Afterwards, the mixture is heated to 60 to 80° C.

2. The fatty components, namely the medium-chain triglycerides, are melted and mixed (“fat composition”).

3. 1 part emulsifier SB is heated with 5 parts “fat composition” of step 2 to 65° C. and is melted until clear, then this mixture is added to the total “fat composition” and is mixed.

4. Fat-soluble components (vit. E, vit. D3, vit. A-palmitate, β-carotene) are diluted therein and mixed.

5. Fat phase and aqueous phase are mixed while stirring at 40 to 50° C. so that an emulsion of the type w/o (water in oil) is formed.

6. The resulting w/o emulsion is crystallised and knead with a scrape surface heat exchanger in the manner known for margarine so as to obtain a product which is easy to spread.

The daily need of margarine containing medium-chain triglycerides is variable, has, however, to be adapted to the specific findings. If the diet margarine is well tolerated, the patient can consume 50 to 70 g thereof per day.

### EXAMPLE 2

### Examination for Determining the Advantageous Effect of Medium-Chain Triglycerides on Overweight or Adiposity

(A) Materials and methods

35 healthy female students of the Karls University in Prague, aged 19 to 24 years and having a body mass index of between 19 and 25 (normal range), participated in the study. They received two controlled diet forms blindly (without knowing the difference between them) ad libitum in three meals spread over the whole day in cross-over design for four weeks each with a washout period of two weeks between the trial periods. The physical weight of the participants was controlled daily.

The ethics commission of the faculty of medicine approved the study. The written consent of the probands concerning their participation in the study was available, containing the commitment not to consume any fat-containing foodstuffs other than the foodstuffs provided. Beverages were permitted and had to be indicated in the daily protocol.

The two diet forms differed in the way the fats contained in the food were composed. The probands of Group A (n=18) firstly received the diet forms with LCT-containing lipids for four weeks. After a two-week break with usual nutrition, the diet form with MCT-containing lipids was consumed for four weeks. Table 1 shows the foodstuffs used for the consumption of MCT-fats. Probands of Group B received the two diet forms at the same times, however, in inverse order.

TABLE 1
Shares in medium-chain and long-chain triglycerides
(MCT/LCT) in specific foodstuffs *) (in 100 g or ml)

| | | MCT % | | LCT % |
|---|---|---|---|---|
| | MCT g | total fat content | LCT g | total fat content |
| Margarine | 66 | 83 | 14 | 17 |
| Cooking oil | 77 | 77 | 23 | 23 |
| Pieces of | 19 | 71 | 7.5 | 29 |
| processed | | | | |
| cheese | | | | |
| Chocolate cream | 35 | 71 | 14 | 29 |
| Turkey paste | 16 | 68 | 8 | 32 |
| Mayonnaise | 40 | 77 | 12 | 23 |
| Muesli bar | 14 | 85 | 2 | 15 |
| (4 types) | | | | |

MCT = medium-chain, saturated triglycerides
LCT = long-chain, saturated and mono/polyunsaturated triglycerides
*) Manufacturer: BASIS Gesellschaft fur Diatetik und Ernahrung mbH, Munich

The preparation of the meals and the weighing of the meals and foodstuffs was done under the control of dieticians in the diet kitchen of the Karls University, lunch (without fat addition) was prepared in the diet kitchen of the Karls University. Lunch was had together, foodstuffs for dinner and breakfast were distributed for taking them home. A nutrition protocol was established for each day. The meals and foodstuffs handed out were weighed; food, which had not been consumed, was deducted. The daily consumption of protein, carbon hydrates, fats (LCT and MCT separately—see Tables 3 to 9) as well as cholesterol, calcium, sodium, potassium, phosphorus, vitamin C and fibres was calculated from the amounts of food which had in fact been consumed. This calculation was done with the help of official food tables and, with respect to the special MCT foodstuffs, in accordance with the manufacturer's indications.

Examples of protocols of the food consumed with regard to the LCT and MCT/LCT diet forms are shown in Tables 3 to 6. The diet was adapted to the nutrition form typical of each country so as to obtain a nutrition form which can be put into practice as easily as possible. Typical foodstuffs and meals are shown in Table 2, as follows.

TABLE 2

| | Typical foodstuffs and meals consumed during the study |
|---|---|
| Break- | Croissant, wheat and rye bread, bread roll, |
| fast | margarine, chocolate cream/Nutella, marmalade, poultry paste, |
| | processed cheese, yoghurt, coffee/tea, apple |
| Lunch | Different kinds of soups (oil), mashed potatoes (oil), pasta (oil), |
| | dumplings, sauce (oil), boiled beef, pork, poultry, vegetables, |
| | cabbage salad, tomato salad, orange |
| Dinner | Croissant, wheat and rye bread, bread roll, poultry |
| | paste, processed cheese, margarine, cucumber salad, |
| | vegetables salad with mayonnaise, fruits, beer/juice. |

TABLE 3
Example of a daily protocol MCT/LCT-diet
Complete meals of March, 29, Proband K P, Group B

| | Amount | Carbohydrates | Protein | Fat | | Energy | |
|---|---|---|---|---|---|---|---|
| Foodstuffs | g/ml | G | G | g MCT | g LCT | kJ | kcal |
| Breakfast | | | | | | | |
| Croissants (3) | 138 | | | | | | |
| Yoghurt | 150 | | | | | | |
| Turkey paste | 25 | | | | | | |
| Margarine | 10 | | | | | | |
| | | 95.8 | 21.4 | 14.0 | 1.5 | 2557 | 611 |
| Lunch | | | | | | | |
| Vegetable soup | 300 | | | | | | |
| with semolina | | | | | | | |
| Beef, boiled | 40 | | | | | | |
| Dill sauce | 130 | | | | | | |
| Semmelnkn?o♭dl | 130 | | | | | | |
| (bread dumpling) | | | | | | | |
| Cooking oil | 20 | | | | | | |
| | | 101.5 | 35.5 | 25.0 | 10.0 | 3643 | 870 |
| Dinner | | | | | | | |
| Croissants (2) | 92 | | | | | | |
| Chocolate cream | 10 | | | | | | |
| Margarine | 5 | | | | | | |
| | | 59.8 | 9.1 | 7.0 | 2.5 | 1528 | 365 |
| Sums | | 257.1 | 66.0 | 46.0 | 14.0 | 7728 | 1846 |
| Break-up of fat in % | | | | 76.7 | 23.3 | | |

TABLE 4
Example of a daily protocol LCT-diet
Complete meals of March, 29, Proband M M, Group A

| | Amount | Carbohydrates | Protein | Fat | | Energy | |
|---|---|---|---|---|---|---|---|
| Foodstuffs | g/ml | G | g | g MCT | g LCT | kJ | kcal |
| Breakfast | | | | | | | |
| Croissants (3) | 138 | | | | | | |
| Poultry paste | 50 | | | | | | |
| Margarine | 10 | | | | | | |
| | | .1 | 17.0 | | 11.9 | 2227 | 532 |
| Lunch | | | | | | | |
| Vegetable soup | 300 | | | | | | |
| with semolina | | | | | | | |
| Beef, boiled | 40 | | | | | | |
| Dill sauce | 120 | | | | | | |
| Semmelnkn?o♭dl | 56 | | | | | | |
| (bread dumpling) | | | | | | | |
| Cooking oil | 15 | | | | | | |
| | | 57.3 | 28.4 | | 29.5 | 2547 | 608 |
| Dinner | | | | | | | |
| Croissants (3) | 138 | | | | | | |
| Margarine | 5 | | | | | | |
| Nutella | 10 | | | | | | |
| | | 90.2 | 13.2 | | 8.2 | 2042 | 488 |
| Sums | | 235.6 | 58.6 | | 49.6 | 6816 | 1628 |

TABLE 5
Example of a daily protocol LCT/MCT-diet
Complete meals of May 10, Proband M M, Group A

| | Amount | Carbohydrates | Protein | Fat | | Energy | |
|---|---|---|---|---|---|---|---|
| Foodstuffs | g/ml | G | g | g MCT | g LCT | kJ | kcal |
| Breakfast | | | | | | | |
| Turkey paste | 25 | | | | | | |
| Yoghurt | 150 | | | | | | |
| Margarine | 10 | | | | | | |
| | | 11.2 | 9.4 | 14.0 | 1.5 | 868 | 207 |
| Lunch | | | | | | | |
| Vegetables | 300 | | | | | | |
| Beef, boiled | 40 | | | | | | |
| Dill sauce | 200 | | | | | | |
| Semmelnkn?o♭dl | 130 | | | | | | |
| (bread dumpling) | | | | | | | |
| Cooking oil | 25 | | | | | | |
| | | 109.3 | 37.1 | 25.0 | 10.0 | 4050 | 967 |
| Dinner | | | | | | | |
| Croissants (2) | 92 | | | | | | |
| Chocolate cream | 10 | | | | | | |
| Margarine | 10 | | | | | | |
| Banana | 80 | | | | | | |
| Orange | 160 | | | | | | |
| | | 101.7 | 10.2 | 10.0 | 4.0 | 2353 | 562 |
| Sums | | 222.2 | 56.7 | 49.0 | 15.5 | 7271 | 1736 |
| Break-up of fat in % | | | | 75.4 | 24.6 | | |

TABLE 6
Example of a daily protocol LCT-diet
Complete meals of May 12, Proband K P, Group B

| | Amount | Carbohydrates | Protein | Fat | | Energy | |
|---|---|---|---|---|---|---|---|
| Foodstuffs | g/ml | G | g | g MCT | g LCT | kJ | kcal |
| Breakfast | | | | | | | |
| Croissants (3) | 138 | | | | | | |
| Yoghurt | 150 | | | | | | |
| Nutella | 20 | | | | | | |
| Margarine | 5 | | | | | | |
| | | 106.9 | 20.5 | | 9.1 | 2470 | 590 |
| Lunch | | | | | | | |
| Vegetable soup with | 148 | | | | | | |
| meat | | | | | | | |
| Pastry with curd | 194 | | | | | | |
| Orange juice | 250 | | | | | | |
| Cooking oil | 12 | | | | | | |
| | | 77.7 | 13.8 | | 23.2 | 2463 | 588 |
| Dinner | | | | | | | |
| Croissants (2) | 92 | | | | | | |
| Processed cheese | 40 | | | | | | |
| Chicken, grilled | 160 | | | | | | |
| Tomato salad | 150 | | | | | | |
| | | 64.5 | 54.0 | | 16.2 | 2477 | 592 |
| Sums | | 249.1 | 88.3 | | 48.5 | 7410 | 1770 |

(B) Results and Discussion

When assessing the protocols concerning the food consumed, it became apparent that during the periods in which MCT/LCT diet was consumed the daily uptake of fat was higher than during the periods in which LCT diet was consumed. Accordingly, the average daily uptake of nutritional energy increased. However, the physical weight in the groups remained, within the usual changes, unchanged despite the higher uptake of energy in the MCT/LTC periods. (See Tables 7 to 9)

TABLE 7
Daily uptake of fat and energy and influence on physical
weight during the study re. consumption of LCT- diet form

| | Fat | Energy | Physical weight | |
|---|---|---|---|---|
| | g/day | KJ/day | kg | Group |
| Start | | | 63.1 | A |
| 1. Week | 59.6 | 8516 | 62.6 | |
| 2. Week | 57.6 | 7965 | 62.4 | |
| 3. Week | 56.4 | 7814 | 62.1 | |
| 4. Week | 58.3 | 8108 | 62.3 | |
| Break/Start | | | 62.5 | B |
| 5. Week | 57.8 | 8022 | 61.8 | |
| 6. Week | 55.8 | 7262 | 61.6 | |
| 7. Week | 56.8 | 7431 | 61.1 | |
| 8. Week | 57.8 | 7246 | 61.2 | |

TABLE 8
Daily uptake of fat and energy and influence on physical
weight during the study re. consumption of MCT/LCT-diet

| | Fat | Energy | Physical weight | |
|---|---|---|---|---|
| | g/day | KJ/day | kg | Group |
| Start | | | 62.9 | B |
| 1. Week | 62.3 | 8374 | 63.3 | |
| 2. Week | 69.6 | 8628 | 62.3 | |
| 3. Week | 71.1 | 8128 | 62.0 | |
| 4. Week | 70.2 | 8132 | 61.8 | |
| Break/Start | | | 62.5 | A |
| 5. Week | 66.1 | 8608 | 62.2 | |
| 6. Week | 68.3 | 8552 | 62.3 | |
| 7. Week | 67.5 | 8230 | 61.9 | |
| 8. Week | 66.2 | 8027 | 61.8 | |

TABLE 9
Average uptake of fat and energy/day and
influence on weight in Part I (week 1-4) and II (week 5-8)

| | | | | Uptake of | | |
|---|---|---|---|---|---|---|
| | | | Uptake of fat | energy | Weight | |
| Group | Part | Diet form | g *) | kJ *) | kg | |
| A | I | LCT | 57.9 | 8069 | Start | 63.1 |
| | | | | | End | 62.3 |
| A | II | MCT-LCT | 67.1 | 8335 | Start | 62.5 |
| | | | | | End | 61.8 |
| B | I | MCT-LCT | 70.1 | 8308 | Start | 62.9 |
| | | | | | End | 61.8 |
| B | II | LCT | 56.0 | 7445 | Start | 62.5 |
| | | | | | End | 61.2 |

*) Total average of individual figures

In both test periods (2×28 days), the daily energy uptake in the diet periods during which MCT was administered was, on average by 565 kJ (60 to 860) higher compared to the diet periods with LCT, without the physical weight being influenced. This could be explained by higher energy consumption due to thermogenesis during MCT administration. In spite of the higher energy administration of approx. 15 MJ in four weeks (which is approximately equal to the uptake of energy from food of 1.5 to 2 days=“fast days”), there were no differences with respect to the probands' weight. Thus, in this study, for the first time, the influence of controlled nutrition with LCT/MCT on weight was examined and documented over a longer period of time (over two months).

Thus, the following conclusions can be drawn from this study:

1. From the results of the study with persons with normal weight, it can be deduced that, when consuming MCT-containing foodstuffs (compared to LCT-containing products), more energy from food can be administered without the physical weight being increased. Thus, increasing the share of MCT-containing foodstuffs in the diet can, with respect to persons with normal weight, be used as a prophylactic measure against overweight.

2. In the case of overweight persons wanting to reduce their weight with the help of a reduction diet (e.g. 1200 kcal/day), the stamina can be increased with MCT-containing foodstuffs—also by more fat as taste carrier—because weight is further reduced despite a higher uptake of energy and thus this seems to be a sensible therapeutic measure for overweight persons.

3. Thus, it follows that it is possible to use MCT-containing foodstuffs for persons with normal weight as well as for overweight persons as a part of a normal or a reduction diet.

1. A method for the prevention or treatment of overweight or adiposity which comprising administering to a patient an effective amount of medium-chain triglycerides or of a composition containing medium-chain triglycerides.

2. The method according to claim 1, wherein the composition comprises a fat phase which contains:
(a) 70 to 90% medium-chain triglycerides,
(b) α-linoleic acid and/or
(c) α-linolenic acid.

3. The method according to claim 2, wherein the composition in the fat phase furthermore contains γ-linolenic acid.

4. The method according to any one of claims 1 to 3, wherein the composition contains 3.8 to 13.4% α-linoleic acid.

5. The method according to any one of claims 1 to 3, wherein the content of α-linolenic acid is 3 to 8%.

6. The method according to claim 3, wherein the composition contains 1 to 2.5% γ-linolenic acid.

7. The method according to claim 2, wherein the content in saturated, long-chain triglycerides in the composition does not exceed 2.5%.

8. The method according to claim 1, wherein the composition in the fat phase has the following composition:

| | |
|---|---|
| medium-chain triglycerides | 70-90%, |
| saturated, long-chain triglycerides | 0.9-2.5%, |
| α-linoleic acid | 3.8-13.4%, |
| α-linolenic acid | 3-8%, |
| γ-linolenic acid | 1-2.5%, |
| oleic acid | 1.3-3.6%. |

9. The method according to claim 8, wherein the composition in the fat phase is composed of:

| | |
|---|---|
| medium-chain triglycerides | 80%, |
| saturated, long-chain triglycerides | approx. 1.6%, |
| α-linoleic acid | approx. 9%, |
| α-linolenic acid | approx. 5.4%, |
| γ-linolenic acid | approx. 1.8%, |
| oleic acid | approx. 2.2%. |

10. The method according to any one of claims 3, 8 and 9, wherein the composition in the fat phase furthermore contains emulsifiers, fat-soluble vitamins, β-carotene and/or lecithin.

11. The method according to claim 10, wherein the fat-soluble vitamins are vitamins A, D and/or E.

12. The method according to claim 2, wherein the fat phase of the composition accounts for approx. 80% and the aqueous phase for approx. 20%.

13. The method according to claim 12, wherein the aqueous phase of the composition contains vitamin C, folic acid and/or vitamin B12.
